(19) United States
(12) Patent Application Publication
(10) Pub. No.: US 2009/0149768 A1
(43) Pub. Date: Jun. 11, 2009

(54) Method for monitoring a person being examined

(75) Inventor: Katrin Christel Sprung, Erlangen (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 12/313,570

(22) Filed: Nov. 21, 2008

(30) Foreign Application Priority Data
Dec. 7, 2007 (DE) 10 2007 059 149.9

(51) Int. Cl. A61B 5/0402; A61B 6/00

(52) U.S. Cl. 600/523; 600/476

(57) ABSTRACT

The invention relates to a method for recording image data of a person being examined in medical diagnostic equipment, comprising the following steps: recording measurement data to create image data; recording at least one of the two variables: heart activity and breathing activity of the person being examined, during recording of the measurement data; automatic analysis of the recorded activity characteristic by comparison with a predetermined activity characteristic; and informing an operator of the medical diagnostic equipment if the analyzed activity characteristic does not match the predetermined activity characteristic.

### CROSS REFERENCE TO RELATED APPLICATIONS

This application claims priority of German application No. 10 2007 059 149.9 filed Dec. 7, 2007, which is incorporated by reference herein in its entirety.

### FIELD OF THE INVENTION

The present invention relates to a method for recording image data of a person being examined in medical diagnostic equipment. The invention is used in particular in examinations in magnetic resonance systems to generate MR image data.

### BACKGROUND OF THE INVENTION

The cooperation of the person being examined is very important when generating MR image data. In addition to the fact that the person being examined is not allowed to move during the measurement, with some measurements it is also necessary for the patient to hold his breath and for the measurement to be coordinated with the cardiac cycle, as is possible for example by way of ECG triggering or pulse triggering. This, in addition to optimum parameter definition of the measurement, is crucial to the resulting image quality.

Once measurement is complete it can be difficult to determine the cause of poor image quality. If the image quality is not satisfactory a measurement has to be repeated, whereby the magnetic resonance system is blocked for longer than necessary by a person being examined.

In the prior art it is known to superimpose the heart activity of the person being examined, for example by way of an ECG (electrocardiogram), during the measurement. It is also known to detect the breathing activity of the person being examined using what is known as navigator measurement or with the aid of a respiration strap. The patient's movement can be followed for example by a monitoring camera which is provided on the MR system.

It is thereby theoretically possible for the operator to check during measurement whether the heart activity is regular or whether the person being examined is breathing regularly or holding his breath.

In practice however the situation often occurs where the operator is already making adjustments for the subsequent measurements or is occupied with other tasks and consequently does not have the displays showing heart or breathing activity constantly in view.

If the person being examined accordingly displays irregularities in his ECG or does not hold his breath for long enough, this can be overlooked and leads to poor image quality. The operator now has the choice of accepting this poor image quality or repeating the measurement. A repetition of the measurement in turn leads to a lower patient turnover.

### SUMMARY OF THE INVENTION

Starting from the above-mentioned drawbacks it is an object of the present invention to optimize recording of image data and monitoring of the person being examined as the image data is being recorded such that certain sources of error are discovered immediately rather than only once the measurement is complete, or such that in the case of poor image quality the cause thereof can be accounted for more accurately.

These objects are achieved by the features of the independent claims. Preferred embodiments of the invention are described in the dependent claims.

According to a first aspect a method for recording image data is provided in which measurement data is recorded in a first step in order to create image data As the measurement data is being recorded either the heart activity or the breathing activity or both of these physiological parameters or vital parameters is/are recorded. The recorded activity characteristic is then automatically analyzed in an additional step by comparison with a predetermined activity characteristic. The operator is informed if the analyzed activity characteristic does not match the predetermined activity characteristic. The operator is assisted with error analysis by automatic analysis of the heart and breathing activity. During measurement the operator can be made directly aware of certain sources of error. If in doubt the operator can stop the measurement immediately and start again once the error has been eliminated if he believes that a measurement with incorrect breathing or heart activity will not provide the desired image quality.

The heart activity can be recorded using an ECG by way of example, it being possible to use the R-wave interval for example to determine the activity characteristic. The breathing activity of the person being examined can be determined using a respiration strap or with the aid of compiled MR image data, for example what is known as navigator technology.

According to a preferred embodiment the breathing activity or the heart activity, or both, is/are stored in conjunction with the compiled image data, so it may subsequently be established whether the heart activity or the breathing characteristic has proceeded in an undesirable manner. In the case of monitoring of the breathing activity this can mean that it is investigated whether a regular breathing characteristic existed; in the case of an MR measurement using the breath-hold technique this means that checks are made throughout the measurement to determine whether the breath was being held. The predetermined activity characteristic in the case of breathing thereby matches either a regular breathing characteristic or a breath-hold characteristic.

When monitoring the heart activity the predetermined activity characteristic means that the heart is beating regularly and there is therefore a regular activity characteristic.

The operator can be informed visually or acoustically, or visually and acoustically, if it is found that irregularities are occurring in the heart or breathing activity. With visual information it is for example possible when displaying the recorded activity characteristic for a deviation from the predetermined activity characteristic to be visually highlighted or marked so the operator quickly sees the irregularity and can decide whether the measurement should be continued or not. If it is possible the image data produced at the recording instant, at which a deviation from the predetermined activity characteristic was detected, can also be immediately displayed to the operator.

It is also possible to automatically propose alternatives for creating image data to the operator if the activity characteristic does not match the predetermined activity characteristic. If irregular heart activity is detected this can mean for example proposing a measurement to the operator in which the measurement data, which was recorded during the irregular heart activity, is not taken into account when compiling the image data. Measuring methods which are not triggered by heart activity, if this is possible, can also be proposed as alternatives.

The breathing or heart activity is preferably recorded for the entire period during which the person being monitored is accommodated in the diagnostic equipment. The regularity of the heart activity or the breathing activity can be determined in that the activity characteristic before recording is analyzed and compared with the activity characteristic during recording of the measurement data. Thus for example an average heart rate can be calculated from n preceding heartbeats, an irregular heart rate being inferred if the current heart rate differs from the average heart rate, calculated from the n preceding heartbeats, by a predetermined value. During the course of measurement the average heart rate can be constantly adjusted to the preceding heartbeats, so it is possible to react to a potentially varying heart rate. A first average value can for example be calculated when three to five heartbeats (N=3 to 5) have been detected. The average breathing activity can likewise be calculated from the preceding breaths before measurement and be compared with the breathing activity during measurement, provided the measurement is not supposed to be recorded using the breath-hold technique.

According to a further embodiment it is also possible to compare the heart activity of the person being examined with predetermined heart activities which may be stored in the diagnostic equipment. These desired heart activities can be used if the heart rate of the person being examined is already so irregular before the start of measuring that significant averaging is not possible. An irregularity is difficult to detect in this case when the heart activity during the measurement is compared with the previous activity characteristic. In this case it may be advantageous to compare the current heart activity with “normal” heart activities stored in advance.

According to a further aspect of the invention continuous checks can be made to determine whether recording of the measurement data has been started, it being possible to store the activity characteristic, recorded during capture of the measurement data, in conjunction with the image data. The activity characteristics determined outside of the period of recording the measurement data can likewise be stored in conjunction with the respective recording instant. The data is therefore preferably stored during and before/after measurement such that at a later instant following measurement it is possible to allocate in terms of time the activity characteristic to measurements that have taken place and measuring breaks.

If information on the manner of the irregularity is produced during detection of the irregularities in breathing or heart characteristic, it can be displayed and also stored with the image data. In addition to displaying the information on the manner of the irregularity, a possible operator's note can be stored with the image data. One possible preconfigured note could for example be:

“Measurement was not repeated as data can be diagnosed”, or

“Measurement was not repeated as person being examined could not tolerate additional measurements”.

Obviously notes produced by the operator himself can also be stored together with the images.

If a recording method using the breath-hold technique is used then this can be detected by the system in advance, the breathing characteristic then being analyzed with regard to whether the person being examined held his breath throughout recording of the measurement data or not.

The invention also relates to medical equipment with an image recording unit for producing image data of the person being examined, a unit for recording the heart or breathing activity, and a processing unit which analyzes the recorded activity by way of comparison with a predetermined activity characteristic. An information unit informs an operator visually or acoustically, or visually and acoustically, if the analyzed activity characteristic does not match the predetermined activity characteristic.

### BRIEF DESCRIPTION OF THE DRAWINGS

The invention will be described in more detail hereinafter with reference to the accompanying drawings, in which:

FIG. 1 schematically shows an MR system with inventive monitoring of the heart and/or breathing activity,

FIG. 2 shows a normal breathing curve with a normal ECG,

FIG. 3 shows a normal breathing curve with irregular ECG,

FIG. 4 shows a normal ECG with normal breathing curve with breath hold,

FIG. 5 shows a normal breathing curve with breath-hold technique and irregular ECG,

FIG. 6 shows a regular ECG with irregular breathing curve,

FIG. 7 shows a regular ECG and a breathing curve in which the person being examined has not held his breath for long enough,

FIG. 8 shows the curves of FIG. 7 with visual highlighting of the error,

FIG. 9 shows a breathing curve with illustrated expiration and inspiration cycles,

FIG. 10 shows an irregular breathing curve with visual highlighting of the irregularity,

FIG. 11 shows a regular ECG displaying the R-wave interval, and

FIG. 12 shows an irregular ECG with visual highlighting of the irregularity.

### DETAILED DESCRIPTION OF THE INVENTION

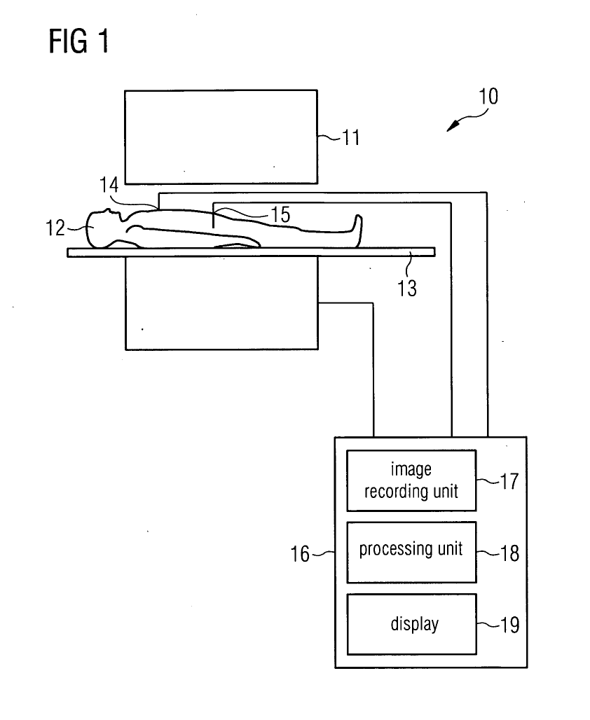

FIG. 1 schematically shows an MR system 10. This MR system has a magnet 11 for generating a polarization field B0, with a person to be examined lying on a couch 13. The magnetization generated in the person being examined 12 can be excited by an HF assembly (not shown) with a high-frequency pulse. With some imaging sequences it may be necessary to monitor the heart activity of the person being examined via ECG electrodes 14. This can be necessary for example in order to control the signal recording in such a way that measurement data is only recorded at a specific instant during the cardiac cycle. It may also be necessary to detect the breathing of a person being examined via a respiration strap 15. The ECG characteristic and the breathing activity can be transmitted to a central control unit 16 which is in turn connected to the MR system for controlling the progression of the examination.

The manner in which a magnetic resonance unit generates an MR image by radiating a sequence of HF pulses and gradients is generally known to a person skilled in the art, so a precise description thereof will be omitted. The central control unit has an image recording unit 17 which controls the sequence with the radiation of the HF pulse and switching of magnetic field gradients. The signals originating from the ECG electrodes 14 and the respiration strap 15 are transmitted to a processing unit 18 which examines these physiological parameters further. If an irregularity is detected by the processing unit an operator (not shown) can be informed of this on a display unit 19.

The processing unit 18 can for example receive the information from the image recording unit 17 as to whether the measurement involves the breath-hold technique. In this case the processing unit 18 can check whether the person being examined 12 holds his breath throughout the measurement.

In other cases the processing unit can check whether the heart rate or the breathing activity was regular.

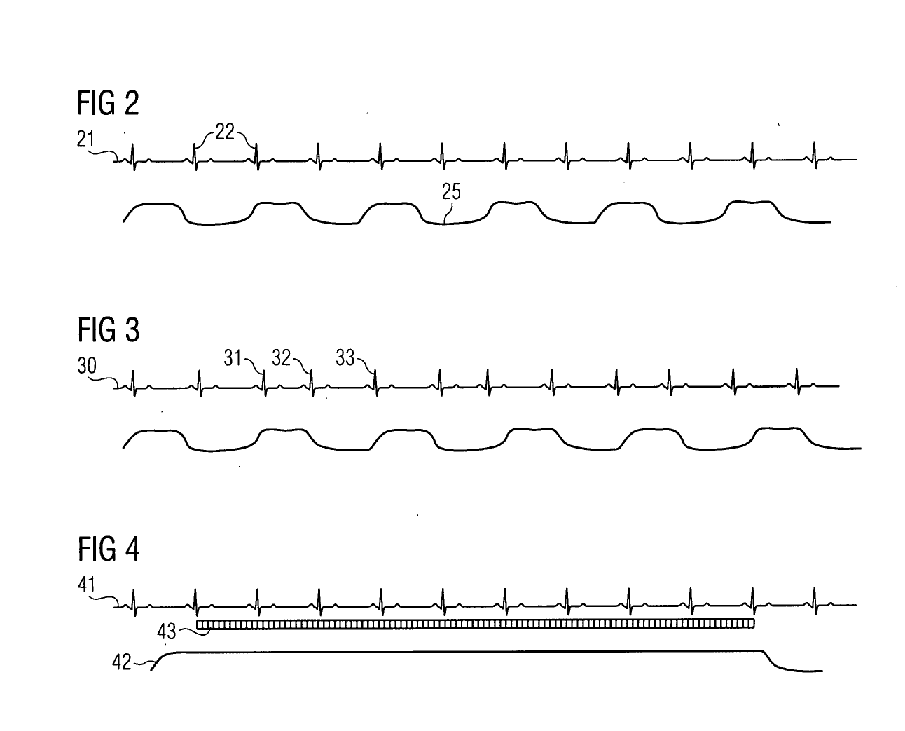

FIG. 2 shows an ECG characteristic 21 and the characteristic of a regular breathing activity 25. The regularity of a heart activity can be determined using the interval between two R-waves 22. In the case of the embodiment shown in FIG. 2 the ECG is regular, as is the breathing activity of the person being examined. The illustrated ECG and breathing characteristics can be stored together with the image data, so the information about the heart activity or breathing activity can also be retrieved at a later date. FIG. 3 accordingly shows how there is an irregular activity characteristic in the ECG signal 30. In the case shown in FIG. 3 the R-wave interval between the R-waves 31 and 32 is for example smaller than the R-wave interval between R-waves 32 and 33. This irregularity can accordingly be graphically displayed and may also be highlighted by an acoustic signal. The user can react and clarify whether it is a general problem, such as poor electrode contact for example, or whether arrhythmia exists, and respond accordingly.

Following measurement, the ECGs can also be analyzed on the MR system or any other computer on which this data is displayed. If arrhythmia exists the system can for example propose alternatives to the operator, for example when recording MR images of the heart the imaging can be controlled in such a way that only measurement data in which there are no irregularities is taken into account, or the measurement data at the irregularity is discarded. It is also possible to change to measurement sequences without heart triggering if the desired information can theoretically be obtained in a recording technique of this kind.

FIG. 4 shows a regular ECG 41 and a breathing characteristic 42 using what is known as the breath-hold technique. The breath-hold technique-means than the breath is held during the measurement data recording 43, symbolically shown by a bar, and this is what the person being examined in the illustrated case manages to do. FIG. 5 shows the same breathing characteristic 42 during measurement data recording 43 but with irregular ECG 51. The breathing curves, as shown in FIG. 2 to 5, can also be monitored and stored with the MR image data.

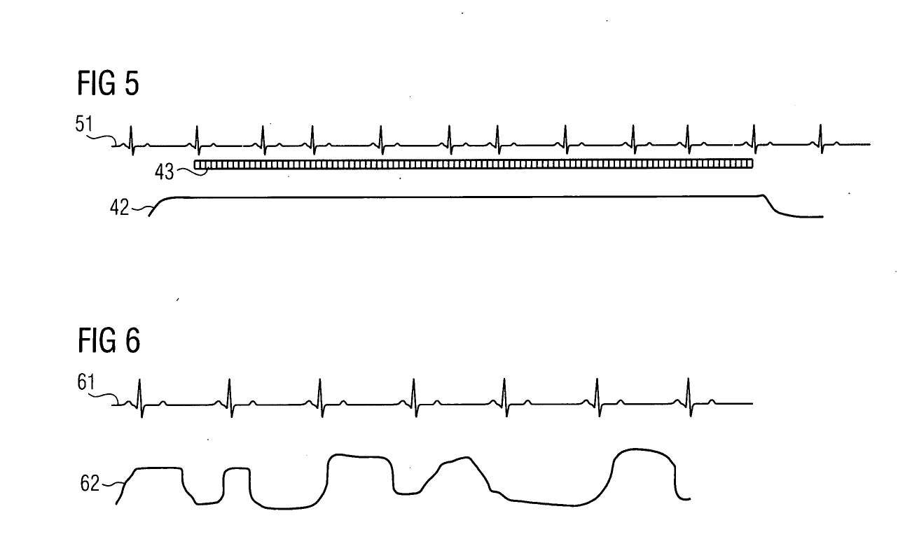

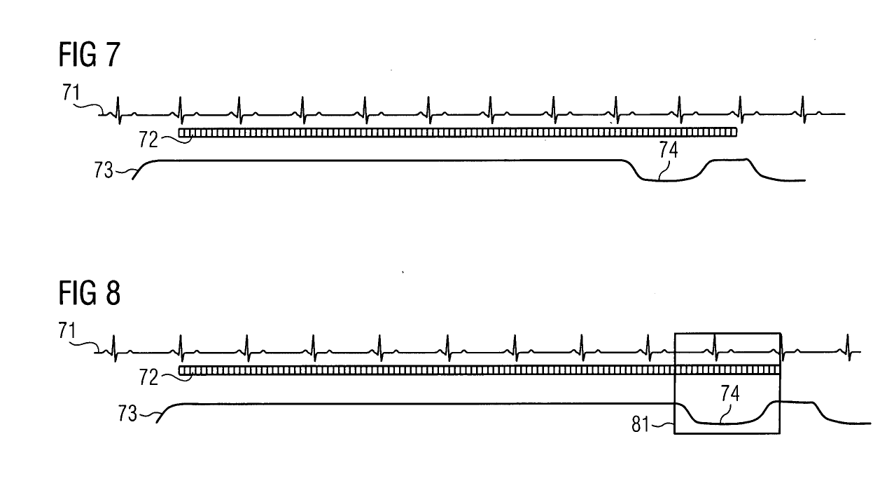

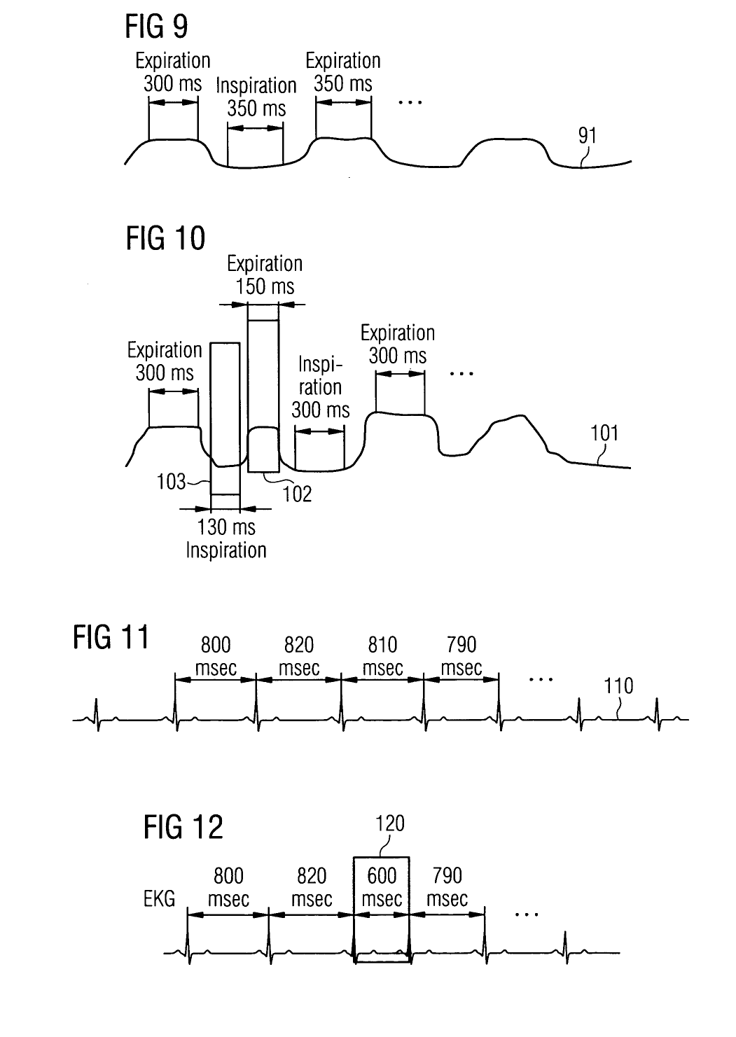

FIG. 6 shows a regular ECG 61 with an irregular breathing curve. If for example the person being examined has breathing difficulties, according to the invention measuring alternatives can be proposed to the operator, for example a measurement with normal breathing or a measurement without breathing triggering. FIG. 7 shows the case where, with a regular ECG curve 71, the person being examined could not hold his breath for the duration of the image acquisition 72, shown by the bar, as can be seen in the breathing characteristic 73. At the end of the measurement the examined person could no longer hold his breath, as is shown by the indentation 74 in the breathing curve caused by breathing in. This error in the breathing activity can, as shown in FIG. 8, be displayed visually enhanced by a frame 81 so the operator is immediately informed that holding of the breath was not successful during the measurement. FIGS. 9 and 10 show a regular breathing characteristic 91 and an irregular breathing characteristic 101 respectively. In the breathing characteristic of FIG. 9 the expiration and inspiration states alternate regularly. This is not the case in the exemplary embodiment of FIG. 10, it being possible to visually highlight the detected irregularity, in this case a relatively short inspiration and expiration time, by the two frames 102 and 103. The ECG or the breathing activity is preferably recorded throughout the period during which the person being examined is lying in the MR system. The activity characteristics can hereby be determined even before the start of the actual MR measurement. The predetermined activity characteristic can be established by examining preceding heartbeats, for example three to five heartbeats. An average value for the R-wave interval can be calculated after three to five heartbeats. The regularity required during measurement can then be assessed using an average value calculated from the preceding intervals. It is possible to determine an average value from the preceding intervals using what is known as a tolerance window. In the ECG characteristic 110 shown in FIG. 11 for example a regular heart activity with an R-wave interval of about 800 ms was determined using a tolerance window. This tolerance window can for example lie at 10%. This tolerance window can of course also have a different value. The processing unit 18 of FIG. 1 determines an average value for the heart activity and analyzes the recorded ECG thereby. If the processing unit finds irregularities, i.e. relevant deviations from an average value in the last-detected interval, this can be highlighted in the ECG curve illustration, as shown in FIG. 12, for example by the frame 120 shown in FIG. 12, to identify a much smaller R-wave interval that lies outside of the tolerance limit.

The image recording unit 17 can also detect whether measurement data is being acquired or not. The heart activity or the breathing characteristic, as shown for example in FIGS. 2 to 12, which was captured during one measurement, can be stored together with the image data and therefore also be displayed again separately or together with the image data. The other activity characteristics, which were captured outside of the measuring periods, can be stored and examined together, the data preferably being stored in such a way that it is subsequently possible to allocate measurements that have taken place and breaks in measuring in terms of time.

If any of the above-mentioned irregularities occurred during the measurement, the operator is also informed of them immediately during or after measurement, for example by indicating the irregularity, this indication automatically being stored with the image data and being displayed therewith. The operator can now examine the activity characteristics with the recording and decide how the additional MR measurement data should be acquired. For example it is possible to automatically propose measuring alternatives, so optimized measurement can take place without time being lost.

If the operator should find that the indication is irrelevant, he can simply remove the indication of the irregularity and when subsequently examined the data appears without indication of irregularities. It is also possible to accept the indication or provide it with a note which is also stored with the image data. Notes that occur frequently could be pre-configured in this connection and be allocated by simply clicking on them. Such notes can for example contain the information that the measurement was not repeated, the MR image data contained the diagnostically relevant information or that an additional measurement was not possible due to lack of patient cooperation.

In the same way the processing unit can determine regular breathing by comparison of breathing before the measurement with breathing during the measurement. Average inspiration and expiration cycles can be calculated and deviations therefrom thus detected. The average value can be adapted to the characteristic of the measurement, so the MR system can adjust to a potentially changing breathing cycle.

If a measurement of MR data is taking place, the processing unit 18 is informed by the image recording unit 17 that the measurement is one using the breath-hold technique. The processing unit can then check whether the breath was held for the duration of the measurement or not. If irregular breathing characteristics are detected, as shown in FIG. 10, these are displayed, it also being possible to propose measuring alternatives to the operator at the same time. One possible alternative would be adjustment of the recording sheet, so it can be combined with the changed expiration and inspiration cycles. Measurement using the breath-hold technique can also be proposed as a measuring alternative.

In order to easily inform the operator about a detected irregularity, cycles which differ from the average value can be marked. Activation or the activation of an acoustic signal when deviating cycles are detected is also possible.

It is also possible to check the breathing characteristic or the heart activity as a whole afterwards, for example by displaying the characteristics with the measuring instants and the instants between the measurement. The operator is therefore capable of commenting on the irregularities seen or discarding them as irrelevant.

1.-23. (canceled)

24. A method for recording image data of a person being examined in a medical diagnostic equipment, comprising:
recording measurement data for creating the image data;
recording a physiological activity of the person being examined during recording the measurement data;
automatically analyzing a characteristic of the recorded activity by comparing with a predetermined activity characteristic; and
informing an operator of the medical diagnostic equipment if the analyzed activity characteristic does not match the predetermined activity characteristic.

25. The method as claimed in claim 24,
wherein the physiological activity comprises a heart activity and a breathing activity, and
wherein the heart activity or the breathing activity or both the heart activity and the breathing activity is recorded during the recording of the measurement data.

26. The method as claimed in claim 25, wherein the heart activity is recorded using an ECG.

27. The method as claimed in claim 25, wherein the breathing activity is determined using a respiration strap or based on the created image data.

28. The method as claimed in claim 25, wherein the heart activity or the breathing activity is stored in conjunction with the created image data.

29. The method as claimed in claim 25, wherein the predetermined activity characteristic matches a regular breathing characteristic or a breath-hold characteristic when monitoring the breathing activity.

30. The method as claimed in claim 25, wherein the predetermined activity characteristic matches a regular heart activity when monitoring the heart activity.

31. The method as claimed in claim 24, wherein the operator of the diagnostic equipment is informed visually or acoustically when an irregularity is detected in the heart activity or the breathing activity.

32. The method as claimed in claim 24, wherein an alternative for creating the image data is automatically proposed to the operator if the analyzed activity characteristic does not match the predetermined activity characteristic.

33. The method as claimed in claim 24, wherein the measurement data that is recorded when an irregularity of the physiological activity is detected is not taken into account when creating the image data.

34. The method as claimed in claim 24, wherein a regularity of the predetermined activity characteristic is determined by monitoring the physiological activity before the recording of the measurement data and comparing the predetermined activity characteristic before and during the recording of the measurement data.

35. The method as claimed in claim 24, wherein an average heart rate is calculated from previous heartbeats and an irregular heart rate is determined if a current heart rate differs from the average heart rate by a predetermined value.

36. The method as claimed in claim 24, wherein a start of the recording of the measurement data is checked.

37. The method as claimed in claim 24, wherein the activity characteristics that are recorded outside of a period of the recording of the measurement data is stored together with a time of the recording.

38. The method as claimed in claim 24,
wherein when an irregularity in the recorded activity characteristic is detected, an indication of a type of the irregularity is displayed and stored with the created image data, and
wherein a note of the operator on the displayed indication is stored with the image data.

39. The method as claimed in claim 24,
wherein before the recording of the measurement data, a technique that is used for the recording is checked, and
wherein the person being examined is checked for whether he holds his breath during the recording if a breath-hold technique is used.

40. The method as claimed in claim 24, wherein a deviation from the predetermined activity characteristic is marked when displaying the recorded activity characteristic.

41. The method as claimed in claim 24, wherein the image data that is created when a deviation from the predetermined activity characteristic is detected at the recording of the measurement data is displayed.

42. A medical diagnostic equipment, comprising:
an image recording unit for producing image data of a person being examined;
a unit for recording a physiological activity of the person being examined;
a processing unit that analyses a characteristic of the recorded physiological activity by comparing with a predetermined activity characteristic; and
a unit that informs an operator of the medical diagnostic equipment if the analyzed activity characteristic does not match the predetermined activity characteristic.

43. An electronically readable storage medium used in a computer for producing image data of a person being examined in a medical diagnostic equipment, comprising:
a computer program for:
recording measurement data for producing the image data;
recording a physiological activity of the person being examined during recording the measurement data;
automatically analyzing a characteristic of the recorded activity by comparing with a predetermined activity characteristic; and
informing an operator of the medical diagnostic equipment if the analyzed activity characteristic does not match the predetermined activity characteristic.
